(19) United States
(12) Patent Application Publication
(10) Pub. No.: US 2014/0273131 A1
(43) Pub. Date: Sep. 18, 2014

(54) METHODS AND SYSTEMS FOR USE IN DEACTIVATING ORGANISMS USED IN BIOPRODUCT PRODUCTION PROCESSES

(71) Applicant: ABENGOA BIOENERGY NEW TECHNOLOGIES, LLC, Chesterfield, MO (US)

(72) Inventor: Matthew Sutton Brandenburg, Ellisville, MO (US)

(73) Assignee: ABENGOA BIOENERGY NEW TECHNOLOGIES, INC., Chesterfield, MO (US)

(21) Appl. No.: 13/800,756

(22) Filed: Mar. 13, 2013

(51) Int. Cl. A61L 2/04; C12M 1/00; C12P 7/06

(52) U.S. Cl. CPC A61L 2/04; C12P 7/06; C12M 47/10
USPC 435/161; 435/289.1; 422/22

(57) ABSTRACT

A method for deactivating organisms used in fermenting a biomass to produce one or more bioproducts (e.g., ethanol, etc.) generally includes an operation of conveying the organisms from a fermentation chamber to a distillation unit, and then an operation of effecting a positive six-log kill of the organisms in the distillation unit. A system for producing the one or more bioproducts, making use of the method, generally includes the fermentation chamber for holding the organisms during the fermentation operation, and the distillation unit configured to receive the fermented biomass, including the organisms, from the fermentation chamber. The distillation unit is operable to effect the positive six-log kill of the organisms in the distillation unit. In some applications of the method and system, the organisms are genetically modified organisms.

### FIELD

The present disclosure generally relates to bioproduct production (e.g., ethanol production, etc.) and, more specifically, to methods and systems for use in deactivating organisms (e.g., naturally occurring organisms, genetically modified organisms, etc.) used to ferment biomass in connection with bioproduct production processes.

### BACKGROUND

This section provides background information related to the present disclosure which is not necessarily prior art.

Bioproducts can be produced from a variety of feedstock materials, including energy crops and cellulosic materials. And typically, production of the bioproducts from the feedstock materials includes pre-treatment (e.g., physical, chemical, enzymatic, etc.) of the feedstock materials, saccharification and fermentation of the pre-treated feedstock materials, and then distillation of the fermented materials to recover the bioproducts.

### SUMMARY

This section provides a general summary of the disclosure, and is not a comprehensive disclosure of its full scope or all of its features.

Example embodiments of the present disclosure generally relate to methods for use in deactivating organisms (e.g., naturally occurring organisms, genetically modified organisms, etc.) used in bioproduct production processes (e.g., ethanol production processes, petrochemical substitute production processes, etc.). In one example embodiment, a method for deactivating organisms used in fermenting a biomass to produce one or more bioproducts generally includes conveying the organisms from a fermentation chamber to a distillation unit, and then effecting a positive six-log kill of the organisms in the distillation unit.

Example embodiments of the present disclosure also generally relate to systems for use in deactivating organisms (e.g., naturally occurring organisms, genetically modified organisms, etc.) used in bioproduct production processes (e.g., ethanol production processes, petrochemical substitute production processes, etc.). In one example embodiment, a system for producing one or more desired bioproducts, and which is capable of deactivating organisms used in fermenting a biomass to produce the one or more bioproducts, generally includes a chamber comprising the organisms operable to ferment the biomass to the one or more bioproducts and a distillation unit configured to receive the fermented biomass (along with the fermentation organisms) from the chamber following completion of the fermentation operation. The distillation unit is operable to separate the bioproducts from the fermented biomass. The distillation unit is also operable to effect a positive six-log kill of the organisms in the distillation unit.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a flow-chart illustrating an example embodiment of a method for use in deactivating organisms (e.g., naturally occurring organisms, genetically modified organisms, etc.) used in a bioproduct production process; and

FIG. 2 is a schematic illustrating an example embodiment of a system for producing one or more bioproducts and utilizing a method for deactivating organisms (e.g., naturally occurring organisms, genetically modified organisms, etc.) used in a fermentation operation of a bioproduct production process used to produce the one or more bioproducts.

Corresponding reference numerals indicate corresponding parts throughout the several views of the drawings.

### DETAILED DESCRIPTION

Example embodiments will now be described more fully with reference to the accompanying drawings.

FIG. 1 illustrates an example method 100 for deactivating organisms used in fermenting a biomass slurry (e.g., for use in producing one or more desired bioproducts, etc.). In various environments, settings, etc. release of active/living fermentation organisms is undesired. The example method 100 provides a positive six-log kill (e.g., sterilization, deactivation, etc.) of the organisms after they are used in fermenting the biomass slurry. As such, the method 100 helps inhibit release of active/living fermenting organisms into the environment, for example, as part of residual fermentation co-product disposal, still bottoms disposal, etc. following the fermenting operation.

The biomass slurry referenced herein can be formed from any desired feedstock or combinations of feedstock. Examples of such feedstock include (without lamination) cellulosic materials (e.g., wood, herbaceous material, agricultural residues, corn fiber, corn stover starch, waste paper, waste cardboard, pulp and paper mill residues, etc.); energy crops (e.g., starch-based crops, etc.) such as cereal grains, corn, sugarcane, oats, wheat, barley, rice, combinations thereof, etc.; municipal solid wastes; other biomass; combinations thereof; etc. The feedstock is initially pre-treated, for example, using suitable physical, chemical, enzymatic, etc. operations (e.g., grinding, heating, steam explosion, acid hydrolysis, enzymatic hydrolysis, combinations thereof, etc.) to form the biomass slurry (and various sugars to be fermented).

The organisms used in connection with fermenting the resulting biomass slurry can include any suitable organisms capable of converting sugars of the biomass slurry to, for example, a desired bioproduct (or multiple desired bioproducts, etc.). For example, the organisms may include naturally occurring organisms such as Saccharomyces cerevisiae (e.g., baker's yeast, etc.), Zymomonas mobilis, Escherichia coli, etc. Or alternatively (or additionally), the organisms may include genetically modified organisms such as, for example, modified versions of Saccharomyces cerevisiae, Zymomonas mobilis, Escherichia coli, other organisms, etc. It should be appreciated that selection of such organisms may depend on the type of feedstock used to form the biomass slurry and/or the type of sugars in the biomass slurry that need to be fermented, etc. And, the desired bioproduct may include ethanol, a petrochemical substitute, any other desired bioproduct, combinations of bioproducts, etc. within the scope of the present disclosure.

As shown in FIG. 1, the illustrated method 100 generally includes an initial operation 104 of fermenting the biomass slurry in a chamber, using the organisms, to produce the desired bioproduct. Next, the illustrated method 100 generally includes an operation 108 of conveying the fermentation organisms from the chamber to a distillation unit, and an operation 112 of then effecting a positive six-log kill of the organisms in the distillation unit. The chamber used in fermenting the biomass slurry can include any suitable chamber (e.g., a vessel, a container, a fermentation chamber, a simultaneous saccharification and fermentation chamber (SSF), combinations of chambers, etc.) capable of accommodating fermentation of the biomass slurry. And, the distillation unit can include any suitable unit capable of separating the bioproduct from the fermentation co-products (e.g., a unit configured to recover a fraction comprising the bioproduct in an overhead stream and the residual fermentation co-products, or stillage, in a bottoms stream; combinations of units; etc.).

The operation 108 of conveying the organisms from the chamber to the distillation unit can include any suitable means operable to move the organisms. For example, tubing, channeling, etc. may be provided between the chamber and the distillation unit. And, gravity, pumps, mechanical drives (e.g., screw drives, etc.), etc. may then be used to move the organisms therethrough. Alternatively, the organisms may be conveyed in bulk from the chamber to the distillation unit (e.g., dumped, scooped, collected in bulk and then moved together, etc.). What's more, the organisms may be conveyed from the chamber to the distillation unit together with the fermentation co-products, or they may be first separated from the fermentation co-products (e.g., in the chamber, etc.) and then conveyed separately to the distillation unit.

The operation 112 of effecting a positive six-log kill of the organisms in the distillation unit generally includes heating the organisms in the distillation unit (e.g., along with the fermentation co-products, etc.) to a temperature of at least about 70 degrees Celsius or more (e.g., about 70 degrees Celsius, greater than about 70 degrees Celsius, about 100 degrees Celsius or more, about 200 degrees Celsius or more, etc.). In addition, in some applications of the example method 100, the operation 112 may further include heating the organisms in the distillation unit to the temperature of at least about 70 degrees Celsius or more for a time period of at least about 1 minute or longer (e.g., about 1 minute, longer than about 1 minute, about 5 minutes or longer, about 10 minutes or longer, about 1 hour or longer, about 2 hours or longer, about 12 hours or longer, etc.). With that said, the inventors hereof have discovered that this application of such heat to the organisms, following use of the organisms in the distillation unit to ferment the biomass slurry to one or more desired bioproducts, will deactivate them (on a six-log level) for safe disposal.

In other example embodiments, methods for deactivating organisms used in fermenting a biomass slurry (e.g., such as the method 100 illustrated in FIG. 1, etc.) may further include one or more additional operations of recovering the deactivated organisms from the distillation unit and of subsequently disposing, recycling, reusing, etc. the deactivated organisms.

As previously stated, the example method 100 illustrated in FIG. 1 can help inhibit the possibility of organisms escaping into the environment (e.g., from the bottom stills of the distillation unit, etc.) after being used to ferment a biomass slurry to a desired bioproduct. This can be advantageous in applications of the example method 100 where release of the fermenting organisms to the environment is scrutinized (e.g., by advocates, by government agencies, by business concerns, by others, etc.). For example, this can be particularly advantageous in applications of the example method 100 where genetically modified organisms are used in fermenting operations (e.g., in applications of the example method 100 where the feedstock includes cellulosic materials, etc.). Here, the method 100 can help avoid the negative impact associated with entry of living genetically modified organisms into the environment, and can help alleviate the scrutiny associated with such use of genetically modified organisms in production processes. Further, the method 100 avoids the need for additional operations typically required to deactivate the organisms (e.g., extreme pH, ultrafiltration, additional heat sterilizers, etc.).

As also previously stated, the example method 100 operates to deactivate organisms used in fermenting a biomass slurry. In so doing, the method 100 provides a positive six-log kill of the organisms following fermentation. As an example, a six-log kill (e.g., sterilization, deactivation, etc.) is the statistical destruction, killing, etc. of all viable organisms and their spores, or a 99.9999 percent reduction thereof. As such, it should be understood that the example method 100 can reduce a population from a million viable organisms to essentially zero viable organisms.

FIG. 2 illustrates an example system 220 for producing a desired bioproduct (or multiple desired bioproducts, etc.). As part of the system 220, a method of the present disclosure is implemented for deactivating organisms used in fermenting a biomass slurry to produce the bioproduct (e.g., in accordance with the method 100 previously described and illustrated in FIG. 1, etc.). As such, the system 220 can operate to provide a positive six-log kill of the organisms after they are used in fermenting the biomass slurry to produce the bioproduct. In turn, the example system 220 also helps inhibit release of active/living fermenting organisms into the environment, for example, as part of still bottoms disposal, etc. following fermentation and distillation operations.

The illustrated system 220 can utilize any desired feedstock to produce the biomass slurry including, for example, lignocellulosic materials, starch-based materials, municipal solid wastes, other biomass materials, combinations thereof, etc. In the illustrated system 220, the feedstock is initially pre-treated in a pre-treatment subsystem 224 using physical, chemical, and/or enzymatic operations (e.g., grinding, heating, steam explosion, acid hydrolysis, enzymatic hydrolysis, combinations thereof, etc.) to form the biomass slurry. This pre-treatment helps clean the feedstock (e.g., helps remove impurities and/or contaminants, etc.) and helps prepare the feedstock for subsequent hydrolysis and fermentation operations.

Next, the biomass slurry from the pre-treated feedstock is transferred (e.g., via pumps and tubing, etc.) to a subsystem 228 where it is subject to hydrolysis (saccharification) and fermentation. The hydrolysis operation helps break down the feedstock and release fermentable sugars (e.g., hexoses, xyloses, arabinoses, etc.) therefrom. This hydrolysis operation may include any suitable operations such as, for example, chemical hydrolysis operations (e.g., steam explosion, acid-catalyzed steam explosion, acid hydrolysis, combinations thereof, etc.), enzymatic hydrolysis operations, combinations thereof, etc. And, the fermentation operation includes converting the released sugars to the desired bioproduct using appropriate organisms (e.g., naturally occurring organisms such as Saccharomyces cerevisiae, Zymomonas mobilis, Escherichia coli, etc.; genetically modified organisms such as modified versions of Saccharomyces cerevisiae, Zymomonas mobilis, Escherichia coli, other organisms, etc.; etc.).

In the illustrated system 220, hydrolysis takes place in a first container at the subsystem 228 and fermentation then takes place in a second container in the subsystem 228. The first and second containers are in fluidic communication such that when hydrolysis is complete, the biomass slurry can move from the hydrolysis container to the fermentation container. The hydrolysis container and the fermentation container can include any suitable container, chamber, vessel, grouping of containers, etc. within the scope of the present disclosure capable of accommodating the hydrolysis and fermentation operations, respectively, of the pre-treated feedstock. With that said, in other example embodiments, systems for producing bioproducts may include combined hydrolysis and fermentation operations (e.g., simultaneous saccharification and fermentation (SSF) operations, etc.) configured to take place in common containers.

Following fermentation, the fermentation co-products are conveyed to a thermal distillation unit 232, via suitable tubing, for distillation. Gravity, pumps, mechanical drives (e.g., screw drives, etc.), etc. may be used to move the fermentation co-products to the distillation unit 232 as desired (through the tubing). The distillation unit 232 is then used to separate the bioproduct from the fermentation co-products. For example, a fraction comprising the bioproduct is recovered in an overhead stream in the distillation unit 232, and the residual fermentation co-products, or stillage, are recovered in a bottoms stream. In the illustrated system 220, the fermenting organisms used to produce the bioproduct are conveyed together with the fermentation co-products to the distillation unit 232 (through the tubing). The distillation unit 232 may include a single unit, or it may include multiple units operated together within the scope of the present disclosure. In other example embodiments, systems may include fermentation and distillation structures where organisms are first separated from fermentation co-products and then conveyed separately to the distillation structures.

The distillation unit 232 is operable to effect a positive six-log kill of the organisms received in the distillation unit 232 (e.g., along with the fermented biomass, etc.). The distillation unit 232 is configured to heat the organisms in the distillation unit 232 to a temperature of at least about 70 degrees Celsius or more (e.g., about 70 degrees Celsius, greater than about 70 degrees Celsius, about 100 degrees Celsius or more, about 200 degrees Celsius or more, etc.). In addition, the distillation unit 232 is configured to retain the organisms in the distillation unit 232 at such temperature for a time of at least about 1 minute or longer (e.g., about 1 minute, longer than about 1 minute, about 5 minutes or longer, about 10 minutes or longer, about 1 hour or longer, about 2 hours or longer, about 12 hours or longer, etc.).

In other example embodiments, methods and/or systems for producing bioproducts may include one or more additional and/or alternative operations and/or structures than illustrated in FIG. 1 or FIG. 2. For example, in some example embodiments, the methods and/or systems for producing bioproducts may include one or more of byproduct recovery and/or treatment operations and/or structures, ethanol recovery and/or processing operations and/or structures (e.g., dehydration processes, molecular sieves, etc.), deactivated organism recovery operations and/or structures, etc.

In one embodiment of the present disclosure, a process for deactivating organisms used in fermenting a biomass slurry includes an initial operation of fermenting the biomass slurry in a chamber, using the organisms, to produce a desired bioproduct, an operation of conveying the fermentation organisms from the chamber to a distillation unit, and an operation of then effecting a positive six-log kill of the organisms in the distillation unit. In this example process, the fermented biomass slurry (e.g., biomass beer, etc.) exits the fermentation chamber and is stored in a well (e.g., a beer well, etc.).

At this point in the process, the fermentation organisms are still viable. The fermented biomass slurry is next drawn from the well and passed through multiple heat exchangers (e.g., three sets of heat exchangers, etc.) that preheat the slurry prior to its introduction into a distillation column (also referred to as a beer column, etc.). The slurry is introduced into the distillation column near an upper portion of the column. The slurry then cascades down the column where it mixes with water and bioproduct vapor rising up the column. The liquid bioproduct (and other light boilers, for example, fusel oils, etc.) along with part of the water in the slurry are evaporated and leave through the upper portion of the column as vapor. In some aspects of the present disclosure, this vapor may then be further purified to produce the desired bioproduct (e.g., ethanol, etc.) as part of the example process. The remaining liquid and solids (including the fermenting organisms) present in the fermented biomass slurry continue to proceed down the distillation column and into a sump. From the sump, the liquid and solids are either drawn off as whole stillage or are recirculated through reboilers and then drawn off as whole stillage. The whole stillage no longer contains bioproducts (or other light boiling components), but does contain large amounts of water and all of the solids (including the deactivated organisms) that entered the distillation column with the fermented biomass slurry. The whole stillage can then be subsequently processed as desired (e.g., reused, recycled, disposed, etc.).

Example embodiments are provided herein so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A method for deactivating organisms used in fermenting a biomass to produce one or more bioproducts, the method comprising conveying the organisms from a fermentation chamber to a distillation unit, and then effecting a positive six-log kill of the organisms in the distillation unit.

2. The method of claim 1, wherein effecting a positive six-log kill of the organisms in the distillation unit includes heating the organisms in the distillation unit to a temperature of at least about 70 degrees Celsius or more.

3. The method of claim 2, wherein effecting a positive six-log kill of the organisms in the distillation unit further includes heating the organisms in the distillation unit to a temperature of at least about 70 degrees Celsius or more for at least about 1 minute or more.

4. The method of claim 1, wherein the organisms are genetically modified organisms configured to ferment the biomass slurry in the fermentation chamber to produce the one or more bioproducts.

5. The method of claim 1, further comprising recovering the deactivated organisms from the distillation unit.

6. The method of claim 1, wherein conveying the organisms from a fermentation chamber to a distillation unit includes pumping the organisms from the fermentation chamber to the distillation unit.

7. The method of claim 1, wherein conveying the organisms from a fermentation chamber to a distillation unit includes conveying the organisms from the fermentation chamber to the distillation unit together with the fermentation co-products.

8. The method of claim 1, further comprising fermenting the biomass in the fermentation chamber.

9. The method of claim 8, wherein the one or more bioproducts include ethanol, and wherein fermenting the biomass in the fermentation chamber includes fermenting the biomass in the fermentation chamber to produce the ethanol.

10. A system for producing one or more bioproducts, the system comprising:
a chamber comprising genetically modified organisms operable to ferment a biomass to a desired bioproduct;
a distillation unit configured to receive the fermented biomass, including the genetically modified organisms, from the chamber, the distillation unit operable to separate the desired bioproduct from the fermented biomass, the distillation unit also operable to effect a positive six-log kill of the genetically modified organisms in the distillation unit.

11. The system of claim 10, wherein the chamber is configured to support simultaneous saccharification and fermentation operations.

12. The system of claim 10, further comprising a pump configured to move the fermented biomass, including the genetically modified organisms, from the chamber to the distillation unit.

13. The method of claim 10, wherein the desired bioproduct includes ethanol, and wherein the distillation unit is configured to separate the ethanol from the fermented biomass.
